(19) United States
(12) Patent Application Publication
(10) Pub. No.: US 2014/0039420 A1
(43) Pub. Date: Feb. 6, 2014

(54) Dr. Courtney Henderson's Cure for HIV of the Soul

(71) Applicant: Courtney Lynette Henderson, Columbia, MD (US)

(72) Inventor: Courtney Lynette Henderson, Columbia, MD (US)

(21) Appl. No.: 13/892,276

(22) Filed: May 11, 2013

Related U.S. Application Data
(60) Provisional application No. 61/645,950, filed on May 11, 2012.

(51) Int. Cl. A61M 35/00

(52) U.S. Cl. CPC A61M 35/00
USPC 604/290

(57) ABSTRACT

A Life-Saving Innovation which utilizes Mind controls/Voice activated Satellite Technology to cure HIV disease of the Soul. Body Scans before and after the process demonstrated that Souls/Agents were cured of HIV The Souls should be a Visible Image. The Satellite—Transforms the Soul into an Invisible Image; performs a series of three Aromatizations; mobilizes materials such as water and a specialized washing solution for the procedure; rinses the Soul with water for two minutes; washes the Soul for three minutes; rinses the Soul for three minutes; performs a series of three Materializations; transforms the Soul into a Visible Image; drys the Soul with heated air at a low velocity for further disinfection. The invention is ten minutes in duration and is usually curative in two treatments; once per consecutive day. Can be performed anywhere in the world. This invention is a “Must-Have.”

### CROSS-REFERENCE

Patent Pending continuous with Provisional Patent: Dr. Henderson's Cure for HIV of the Soul. Application No. 61/645,950. Attorney Docket Number TP-HENC-CHS1. United States Patent and Trademark Office, May 11, 2012.

### THE INVENTOR

Dr. Courtney Henderson was born in the United States of America. Dr. Henderson earned her Post Doctoral credentials in Medicine and was the Valedictorian of her Internship Class of physicians. Dr. Henderson is also a Board Certified Diplomat, and being the highest scoring physician on the Specialty Boards, Dr. Henderson earned her Fellowship credentials.

Currently, Dr. Henderson is utilizing Computational Biology and Aeronautical Engineering to develop inventions with Space Age Technology. Dr. Henderson was highly devoted to rescuing Souls who were being tortured and who were otherwise brutalized in concentration camps. This invention, Dr. Courtney Henderson's Cure for HIV of the Soul, is Dedicated to the endearing Souls who were tortured—this is an Innovation for the Soul.

Dr. Henderson enjoys a plethora of hobbies to include traveling, scuba diving, windsurfing, sailing, yoga, and running. Dr. Henderson is a Certified Open Water Scuba Diver, a former World Class Athlete in Track and Field, and studied yoga at Sivananda Ashram Yoga Retreat in Paradise Island, Bahamas.

### FIELD OF THE INVENTION

The Human Immunodeficiency Virus (HIV) is one of the world's foremost leading threats to the health and the development of most countries. Since the HIV Epidemic of 1981, 34 Million people have been infected with HIV, and approximately 30 Million people have died. The latest tabulations demonstrate that in 2011, there were 2.5 Million new infections, and 7,000 people were infected per day. Therefore, HIV is recognized as an important condition to alleviate. The present invention generally relates to a method for the removal of HIV. More specifically, I developed a process which cures or eliminates HIV disease from the Soul. My Patentable Process is unique. No other person has ever invented a similar process.

My invention has enormous significance which includes uplifting the health of persons and Agents, as well as providing significant socioeconomic gains for individuals, countries, or Agencies. All of the Souls, whom were provided my invention, were cured of HIV. The process is also statistically significant (p<0.001), and the Confidence Intervals are at a 100% confidence level. The statistics demonstrate that the process is causative. My invention must be followed exactly as specified, without deviation to be curative. I based the process on theories developed over a period of time, to produce beneficial results, during a State of Emergency. In the following account, I explain the process in manageable terms. However, the theory behind each step denotes an intricate, complex relationship relating to Physics and Molecular Biology. My philosophy of life relates to a healthy Mind, a healthy Body, and a healthy Soul.

### BACKGROUND OF THE INVENTION

Life-Saving Innovation which utilizes Space-Age Technology to cure HIV disease of the Soul. This is a Patentable Idea and Process to rid contaminants such as HIV disease from the Soul. The Soul is the same entity described in Religious doctrines and by the Central Intelligence Agency. The invented—Process removes the Human Immunodeficiency Virus (HIV) from the Soul, leading to a decrease in the HIV count and a total cure. This Revolutionary Process should decrease the prevalence and transmission of HIV disease. The longevity of the Soul or the Agent should increase without having HIV disease. Infection as a cause of death is usually NOT reversible, so Time Reversal is usually NOT applicable with infections. Therefore, making my Invention essential for survival. The problem is the fatal or the ominous HIV disease. The Invention solves the problem of contracting and transmitting HIV. The Invention also eliminates pre-existing HIV disease. The significance is that there are world-wide implications of eradicating HIV of the Soul.

### BRIEF DESCRIPTION OF THE DRAWINGS

All illustrations, photographs, and drawings are for the purpose of describing selected versions of the present invention and are not intended to limit the scope of the present invention.

FIG. 1 is a front-side illustration showing a “Sunshine” network of satellite systems, representing world-wide range.

FIG. 2 is a diagram of a scientific satellite communication of the present invention.

FIG. 3 is a front-side illustration of the physical separation of molecules of the present invention.

FIG. 4 is an illustration of a scientific satellite in orbit, with electromagnetic signals to an Earth station of the present invention. Earth station elevation angles demonstrate the range and plane of signals.

FIG. 5 is a front-side photograph of the transporter system concept for aromatization of invisible images of the souls of the present invention.

FIG. 6 is a profile demonstrating a representation of an aromatized head shot of the soul.

FIG. 7 is a back-side photograph showing an .aromatized full body shot of a representation of the soul.

FIG. 8 is a front-side illustration of a rinse-wash-rinse section of the present invention.

FIG. 9A is a representation showing materialization of one molecule of the soul.

FIG. 9B is a profile representing materialization of the soul.

FIG. 10 is a front-side photograph of a logo of the present invention.

### DETAILED DESCRIPTION OF THE INVENTION

The present invention is a method to cure HIV disease from the Soul. The Patentable Process is a novel approach utilizing satellite technology. Body scans demonstrated that the Soul and/or Agents were cured of HIV. A body scan was performed before and after the process to provide an HIV count. The satellite Invisibilizes the Souls and mobilizes materials for a procedure. No plumbing or faucets are needed; the water and a specialized washing solution appear “out-of-thin air” and can be performed anywhere in the world. The process can even be performed outside. No special laboratory is required. The process is efficient and effective and is approximately ten (10) minutes in duration. The process eliminates HIV usually in two (2) treatments.

The present invention is relevant to Agencies, such as the Central Intelligence Agency (CIA), the National Aeronautics Space Administration (NASA), the Military, and Business Intelligence Companies. I postulated that HIV disease may be contracted by an Agent from the study subject due to exposure of infectious bodily fluids. Furthermore, I have postulated that this route of transmission is significant and should be included in the scientific literature with the other routes of transmission. I postulated that the Agent may also transmit HIV to the stud_{y} subject and infect the subject. Additionally, it has been witnessed that once the Soul or the Agent contracts the disease, the Soul or the Agent can then spread the disease to his or her own body when reassembled, as well as infect other close contacts, who come in contact with his or her infectious bodily fluids. By providing the Patentable Process to all Agents, before and after the assignment, HIV disease can be eradicated. The present invention also helps alleviate pain and suffering in Souls who are otherwise not Agents, but who have been afflicted with the disease

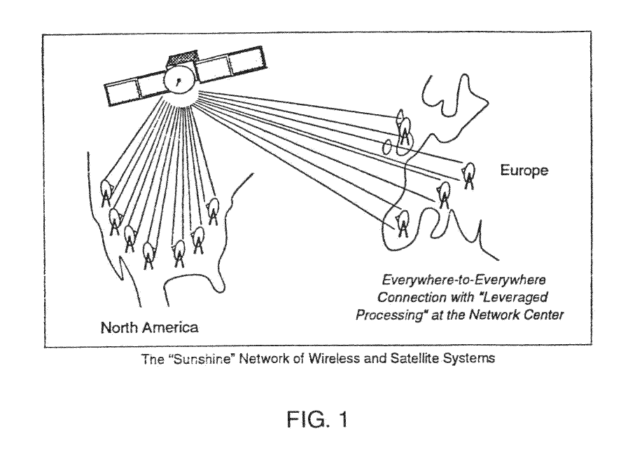

The present invention uses the ultimate in technology and is Space-Age. The present invention does not involve medicine or pills. The process takes approximately ten (10) minutes in duration, which is an enormous decrease in time involvement for treatment. The present invention is usually successful in only two (2) treatments and can be applied anywhere in the world, as seen in FIG. 1

The present invention can be used by various groups, businesses, and individuals world-wide. The present invention can be used by individual Souls worldwide, by CIA Agents, by NASA Agents, by soldiers, and by Business Intelligence companies. Agencies would likely purchase the present invention over an individual person, due to the high-tech equipment involved and expense.

The present invention has numerous benefits to users. The present invention should promote good health, longevity, and better sanitation and living standards. Survivors have also shown increased productivity after receiving the process as described in the present invention.

### IDENTIFICATION OF THE PARTS/COMPONENTS OF INVENTION

Reference Numeral—Descriptive Item for Part
10—Mind controls
12—Voice controls
14—Remote control device/Computer controls
16—Electromagnetic signals from control to satellite
18—Scientific Satellite
20—Electromagnetic signals from the satellite to the Earth Station or Ground Station
22—Electromagnetic signals from the satellite to the Souls
24—Souls
26—Water
28—Washing Solution
30—Air
32—Electromagnetic signals and Materials from the Ground Station to the Souls
34—Time Reversal Applications, optional

### DESCRIPTION OF THE PARTS OF THE INVENTION

10. Mind controls:

Implanted in the brain of the Inventor

This Inventor's CIA IQ (Intelligence Quotient): 212

12. Voice controls:
The satellite responds to Voice Recognition
   14. Remote control device or Computer:
An alternative to Mind controls and Voice controls
   16. Electromagnetic signals from control to satellite

### 18. Scientific Satellite:

‘A satellite in general is an artificial body moving around a celestial body such as planets and stars.’ This Scientific Satellite is Intelligent and continues to learn. Programming by Inventor using Mental telepathy or Voice recognition via normal spoken language in English, Boolean Algebraic language, or traditional computer language. This particular Scientific Satellite is: square in shape, not circular in shape. Several square compartments with hinged doors and locks. Flat platform.
   20. Electromagnetic signals from the satellite to the Ground Station
   22. Electromagnetic signals from the satellite to the Souls:
To transform the Souls—Visible to Invisible Images; Aromatizations; Materializations.

### 24. Souls:

Interior Jelly Liquid of Human Beings, comprised mostly of nervous tissue and major organs such as a Soul brain, a heart, the lungs, and the kidneys. The personality of an individual. Responsible for several essential functions and tasks.

### 26. Water:

Used to Rinse the Souls before and after the Wash. Clean water. No other specifications required such as purified or distilled. The temperature of the water is lukewarm, around 60 degrees Fahrenheit.

### 28. Washing Solution:

A specialized scientific solution; the NASA Scientific Solution.

### 30. Air:

The Souls are dried for two minutes using warm air blowing at a low velocity. A room filled with circulating air.
   32. Electromagnetic signals and Materials from the Ground Station to the Souls

### Description of the Overall Structure of the Invention

Patentable Idea and Process: A series of steps or a procedure involving Satellite Technology.

### Relationship Between the Parts of the Invention

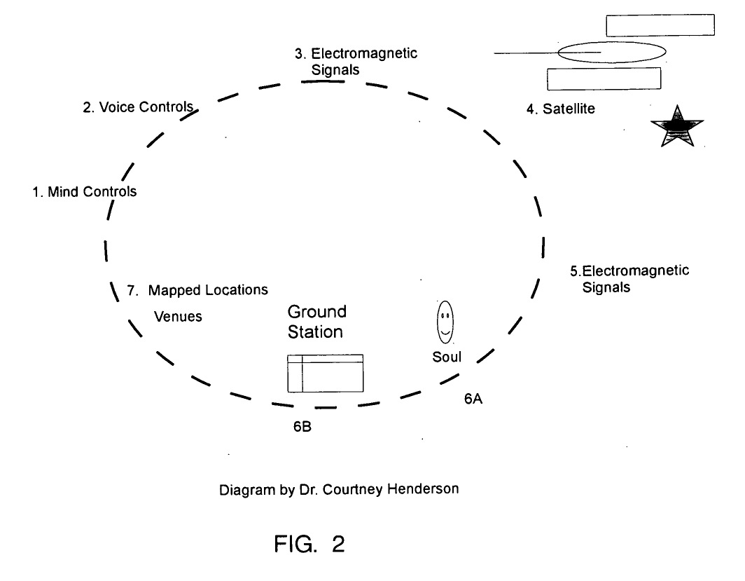

As seen in FIG. 2, Mind controls (1) and Voice controls (2) send commands or electromagnetic signals (3) to the Satellite (4) which send the signals back to Earth. The signals travel to the Soul (6A) and then the signals travel to a Ground Station (6B) to mobilize materials to engage (7).

### DESCRIPTION OF HOW THE INVENTION OPERATES/FUNCTIONS

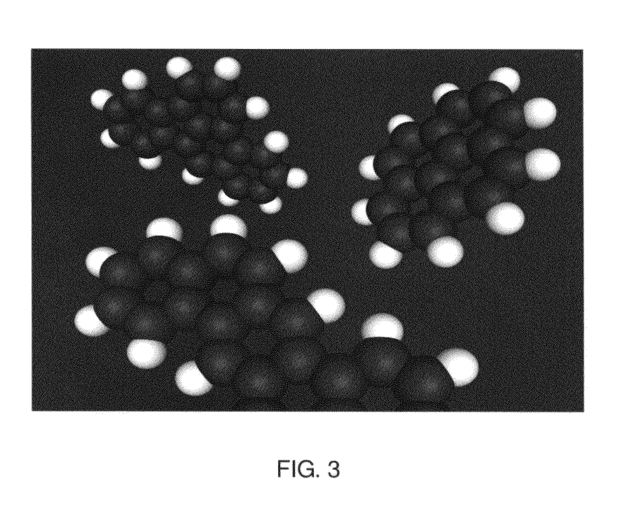

The present invention involves a process to “wash” the soul free of contaminants and pathogens. The novel aspect of the present invention is that the “washing process” is curative of pathogens such as the Human Immunodeficiency Virus (HIV). The Process involves forming an Invisible Image (an image having a form which cannot be perceived by unaided vision, such as a latent image on a photographic emulsion), a series of Aromatizations [1. Disassembling molecules] as seen in FIG. 3, a series of Materializations [1. The direct conversion of energy into mass, as in pair production or the conversion of a photon into an electron and a positron when the photon traverses a strong electric field, such as that surrounding a nucleus or an electron. 2. Reassembling molecules], and Disinfection using heated air at a low velocity. The present invention operates with electromagnetic signals from a remote source such as Mind controls or Voice controls, which transmit commands in the form of electromagnetic signals to a scientific satellite orbiting the Earth.

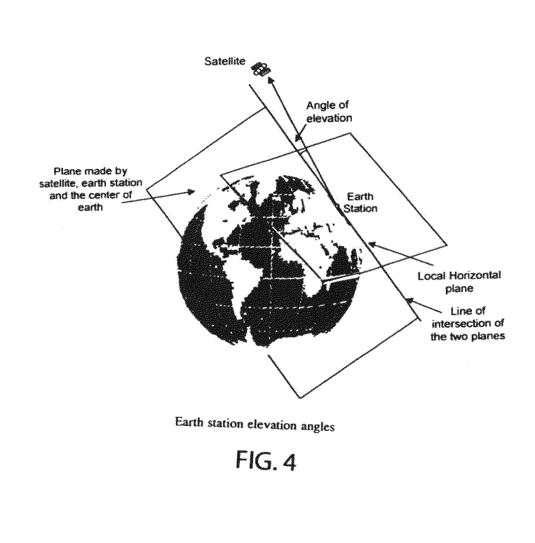

The scientific satellite then signals to the Soul to change the image of the Soul from Visible to Invisible. The scientific satellite also signals an Earth Station to mobilize materials for the Process as seen in FIG. 4. The present invention is for restoration of the SOUL ONLY. WARNING: The present invention is NOT to be used on the entire body, especially NOT on the cranial region of the human body.

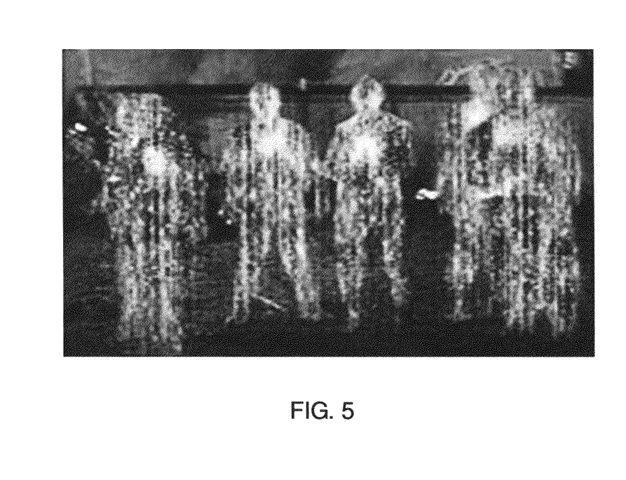

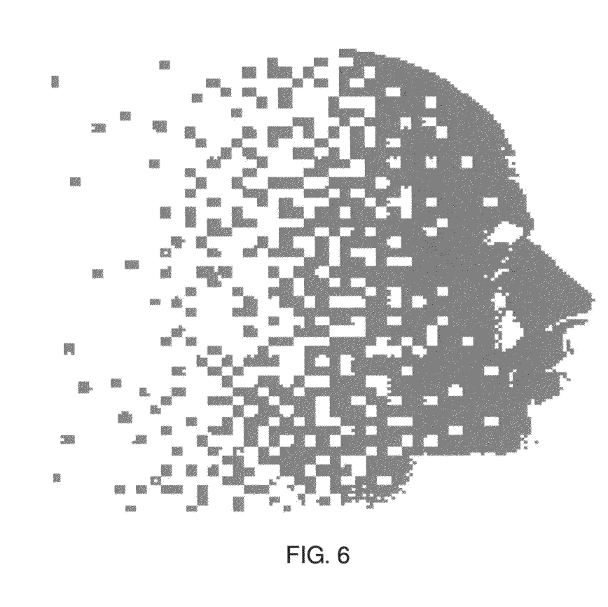

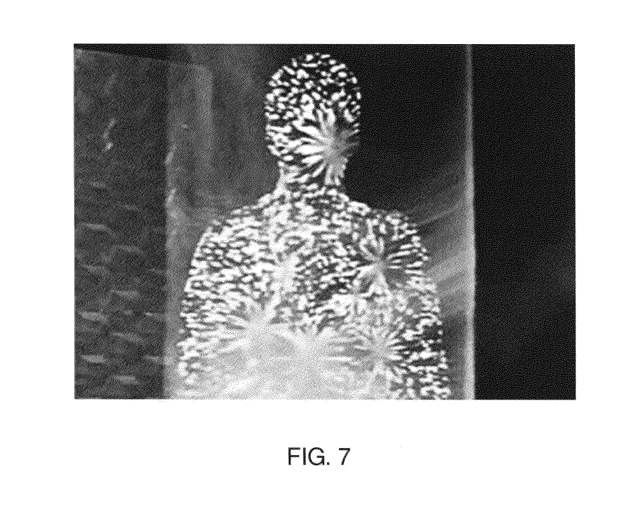

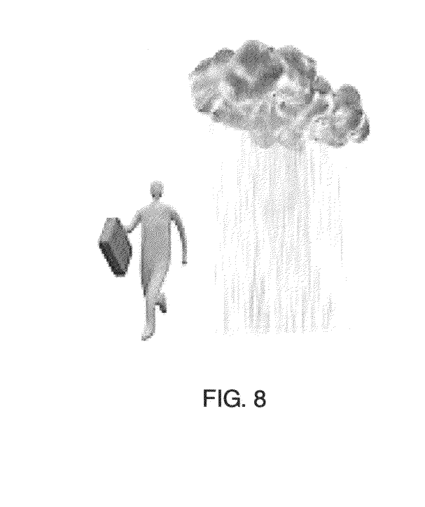

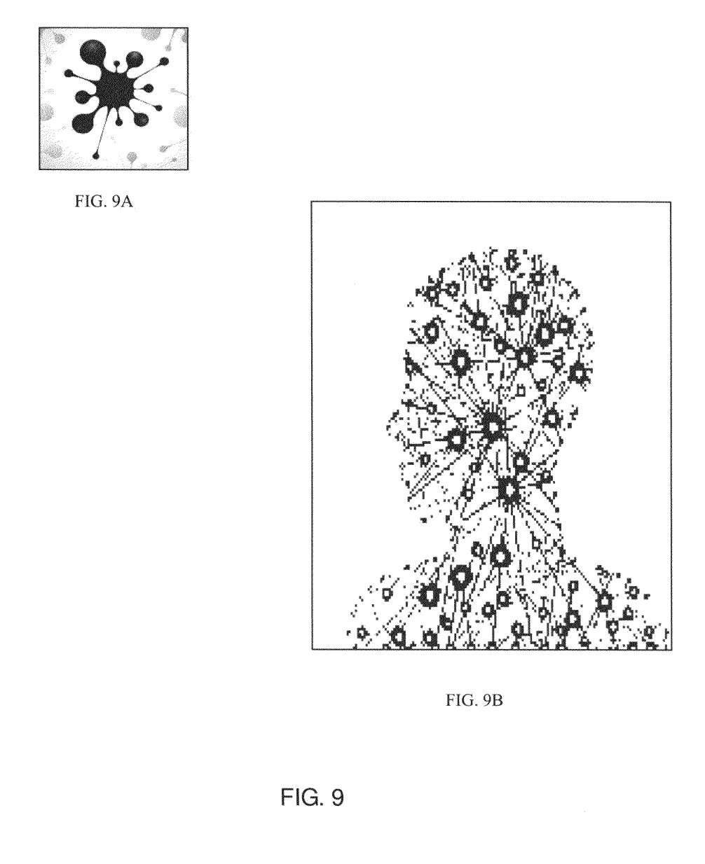

The Process is approximately ten (10) minutes in duration. The process should only be performed once daily. The Process is usually curative of HIV in two (2) days. Further steps of the Process are as follows:
1. Perform a Body Scan on the Soul to count HIV.
2. The Soul should be a Visible Image.
3. Elevate the Soul three (3) feet higher such that the feet do not touch the floor. Perform in an area where Biohazards can be contained, as the floor may become contaminated.
4. Transform the Soul into an Invisible Image as seen in FIG. 5.
5. Aromatize the Soul as seen in FIG. 6.
6. Aromatize the Soul as seen in FIG. 7.
7. Aromatize the Soul.
8. Rinse the Soul with water for two (2) minutes; rinsing from the head toward the toes, using a shower of water traveling at a moderate rate.
9. Wash the Soul for three (3) minutes; the scientific satellite signals the Ground Station for the washing solution to be applied, and the satellite signals for the solution to be agitated throughout the Soul.
10. Rinse the Soul for three (3) minutes as seen in FIG. 8.
11. Materialize the Soul as seen in FIG. 9.
12. Materialize the Soul.
13. Materialize the Soul.
14. Transform the Soul to a Visible Image
15. Command the Satellite to remove contaminants from under the Soul or to move the Soul laterally and then place the Soul on the clean floor.
16. Command the Satellite to dry the Soul for two (2) minutes using heated air or wind circulating at a low velocity for further disinfection.
17. Perform a Body Scan of the Soul to re-evaluate the HIV count.

### Unique Features of Invention

The present invention provides a radiant appearance due to the cleansing, both externally and internally. The present invention provides a Nouveau, State-of-the Art alternative to scrub tanks which were traditionally used to cleanse the outside of the Soul, and the scrub tanks way actually cross contaminate a Soul. The present invention does not require bulky equipment and can be used anywhere as long as the Biohazards are contained or cleaned to OSHA specifications.

### Alternative Embodiment of this Invention (Optional)

1. Computer controls instead of Mind controls or Voice controls
2. NASA Scientific Satellite or a CIA Scientific Satellite
3. Time Reversal Applications are not applicable

Infection is usually not reversible, so Time Reversal is usually not applicable with infections. Therefore, making the present invention essential for survival.

### Raison D'etre

### Dedication

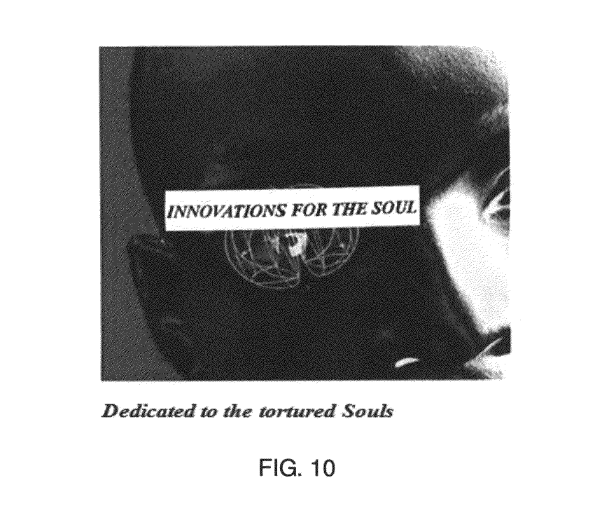

This Invention and ALL of my Love is DEDICATED TO . . . All of my beloved Souls . . . All of my Moms, Dads, Sisters, Nephews, Cousins, Grandmothers, Grandfathers, and my very own Souls as seen in FIG. 10.

### REFERENCES CITED

Anil, K., Agrawal, Varsha. Satellite Technology Principles and Applications. West Sussex, England: John Wiley and Sons, Ltd, 2007.

Godwin, Robert and Whitfield, Steve, eds. Deep Space the NASA Mission Reports. Ontario: Apogee Books, 2005.

Henderson, Courtney, M.D. FIG. 2. 2013.

Henderson, Courtney, M.D. Authorship/Graphic enhancements of black and white drawings: FIG. 3. FIG. 4. FIG. 5. FIG. 6. FIG. 7. FIG. 8. 2013.

Henderson, Courtney, M.D. Authorship/Graphic enhancements of color drawings: FIG. 6. FIG. 8. FIG. 10. 2013.

McGraw-Hill Dictionary of Scientific and Technical Terms. 6^{Th} edition. New York: McGraw-Hill, 2003.

Pelton, Joseph. Wireless and satellite telecommunications, the market & the regulations. Upper Saddle River, N.J.: Prentice Hall PTR, 1995.

Todd, Robert W., et al., eds. Physical Science. Austin, Tex.: Holt, Rinehart and Winston, 2001.

Elmedany, Ahmed (2011). Harvard Reference examples. http://www.slideshare.net. (5 May 2012).

The Henry J. Kaiser Foundation (2012). The Global HIV/AIDS Epidemic. http://kff.org/global-health-policy/fact-sheet/the-gloal-hivaids-epidemic/. (7 Aug. 2013)

Lavender, Catherine (2010). How to Cite, Bibliography: Using the Chicago Manual of Style. http://www.librarv.csi.cuny.edu. (21 Aug. 2013)

Biotechnology. http://newsdiscovery.com. (2 May 2012)

Confidence Intervals. http://www.stat.vale.edu. (13 Aug. 2013)

How can I make a bibliography of a website. http://au.answers.yahoo.com. (5 May 2013)

How to Calculate P value. www.wikihow.com/Calculate-P-Value. (13 Aug. 2013)

http://donaldsweblog.blogspot.corn. (2 May 2012)

http://thinkstockphotos.com. (2 May 2012)

http://www.en.wikipedia.org/wiki/polycyclic_aromatic hydrocarbon.(2 May 2012)

1. This invention, Dr. Courtney Henderson's Cure for HIV of the Soul, is a Novel, Innovative, and Non-Obvious Invention which truly works. Dr. Henderson's ideas are genuine, and Dr. Henderson performed the Engineering and perfected the invention. Dr. Henderson's Invention can NOT be modified NOR can variations be made without consulting Dr. Henderson regarding her expertise. Danger may result if modifications and variations are applied without the proper experience or educational background in Computational Biology and/or Aeronautical Engineering. Dr. Courtney Henderson's Cure for HIV of the Soul usually works within two “washings” and should be used judiciously, not over-zealously. If the process is not followed, as specified, overdoseage or a drowning effect may occur. Judicious applications each on separate days are curative. This invention was invented and dedicated just for the Soul . . .
